(19) United States
(12) Patent Application Publication
(10) Pub. No.: US 2025/0139742 A1
(43) Pub. Date: May 1, 2025

(54) VIDEO PROCESSING SYSTEM, MEDICAL INFORMATION PROCESSING SYSTEM, AND OPERATION METHOD

(71) Applicant: SONY GROUP CORPORATION, TOKYO (JP)

(72) Inventors: TAKESHI MIYAI, TOKYO (JP); KENTARO FUKAZAWA, KANAGAWA (JP)

(21) Appl. No.: 18/693,560

(22) PCT Filed: Sep. 21, 2022
(86) PCT No.: PCT/JP2022/035092

§ 371 Date: Mar. 20, 2024

(30) Foreign Application Priority Data
Oct. 1, 2021 (JP) 2021-162702
Nov. 1, 2021 (JP) 2021-178867

(51) Int. Cl. G06T 5/60; G06T 5/50; G06T 7/00; G06T 7/90; G06T 11/00

(52) U.S. Cl. CPC G06T 5/60; G06T 5/50; G06T 7/0014; G06T 7/90; G06T 11/001; G06T 2207/10016; G06T 2207/10024; G06T 2207/20081; G06T 2207/20084; G06T 2207/30004; G06T 2210/41

(57) ABSTRACT

The present disclosure relates to a video processing system, a medical information processing system, and an operation method capable of obtaining a video with intended image quality. A first medical video captured by a target medical apparatus, which is a first medical apparatus and a second medical video captured by a subject medical apparatus, which is a second medical apparatus are recorded in a video recording unit. Then, a conversion parameter generation unit generates a conversion parameter that brings the image quality of the second medical video close to the image quality of the first medical video by using the first medical video and the second medical video, and an image quality conversion processing unit converts the image quality of a medical video captured by the subject medical apparatus by using the conversion parameter. The present technology can be applied to, for example, a video processing system for a medical institution.

### TECHNICAL FIELD

The present disclosure relates to a video processing system, a medical information processing system, and an operation method, and more particularly, to a video processing system, a medical information processing system, and an operation method capable of obtaining a video with target image quality.

### BACKGROUND ART

Conventionally, when a new medical apparatus is introduced in a medical institution such as a hospital or a clinic, there has been a demand for adjustment so that the image quality does not change from the image quality of a video of another medical apparatus that has been used so far so that a medical practice can be performed with the new medical apparatus in a manner similar to the conventional manner. Therefore, adjustment has been performed so that the image quality of a video captured by a first medical apparatus (hereinafter referred to as a subject medical apparatus) to be subjected to image quality adjustment approaches the image quality of a video captured by the target second medical apparatus (hereinafter referred to as a target medical apparatus).

For example, such image quality adjustment is performed by a person comparing a video of the target medical apparatus and a video of the subject medical apparatus, which takes time and effort, and moreover depends on the sense of the person. Therefore, the accuracy is lowered. Therefore, a technique of adjusting image quality by calculating or correcting a parameter by a device has been proposed.

For example, Patent Document 1 proposes a technique in which a test signal for characteristic measurement is input from a test signal generator to both a device to be subjected to adjustment and a target device to acquire characteristic data, and a conversion parameter that brings image quality of the subject device close to image quality of the target device is calculated using the characteristic data.

Furthermore, Patent Document 2 proposes a technique in which an image to be a target of image reproduction is input in image processing, the image is analyzed to extract a tendency of image reproduction and correct a parameter of the image processing, and the image processing is performed by using the corrected parameter.

### CITATION LIST

### Patent Document

Patent Document 1: Japanese Patent Application Laid-Open No. 2008-199118
Patent Document 2: Japanese Patent Application Laid-Open No. 2006-80746

### SUMMARY OF THE INVENTION

### Problems to be Solved by the Invention

As described above, although a technique of adjusting image quality has been proposed in the related art, it is required to obtain a video having intended image quality by using as little manual labor as possible.

The present disclosure has been made in view of such a situation and an object thereof is to obtain a video with intended image quality.

### Solutions to Problems

A video processing system according to a first aspect of the present disclosure includes: a video recording unit that records a first medical video captured by a target medical apparatus, which is a first medical apparatus and a second medical video captured by a subject medical apparatus, which is a second medical apparatus; a conversion parameter generation unit that generates a conversion parameter which brings image quality of the second medical video close to image quality of the first medical video by using the first medical video and the second medical video recorded in the video recording unit; and an image quality conversion processing unit that converts image quality of a medical video captured by the subject medical apparatus by using the conversion parameter.

In the first aspect of the present disclosure, the first medical video captured by the target medical apparatus, which is the first medical apparatus and the second medical video captured by the subject medical apparatus, which is the second medical apparatus are recorded, the conversion parameter which brings image quality of the second medical video close to image quality of the first medical video is generated by using the first medical video and the second medical video, and image quality of a medical video captured by the subject medical apparatus is converted by using the conversion parameter.

A medical information processing system according to a second aspect of the present disclosure includes: one or more processors; and a storage device that stores a program executed by the one or more processors, in which the program is executed by the one or more processors to read a parameter conversion rule determined in advance, the parameter conversion rule determined in advance being set on the basis of comparison between a pair of images including an image of a second medical apparatus subjected to image quality conversion processing of bringing image quality close to image quality of an image of a first medical apparatus and an image of the second medical apparatus, and to perform parameter conversion processing of converting image quality on an image of the second medical apparatus that has been input on the basis of the parameter conversion rule determined in advance.

An operation method of a medical information processing system according to the second aspect of the present disclosure, the medical information processing system including one or more processors, and a storage device that stores a program executed by the one or more processors, the operation method includes:
by executing the program by the one or more processors,
reading a parameter conversion rule determined in advance, the parameter conversion rule determined in advance being set on the basis of comparison between a pair of images including an image of a second medical apparatus subjected to image quality conversion processing of bringing image quality close to image quality of an image of a first medical apparatus and an image of the second medical apparatus; and
performing parameter conversion processing of converting image quality on an image of the second medical apparatus that has been input on the basis of the parameter conversion rule determined in advance.

In the second aspect of the present disclosure, by executing the program by the one or more processors, the parameter conversion rule determined in advance, the parameter conversion rule determined in advance being set on the basis of comparison between the pair of images including an image of the second medical apparatus subjected to image quality conversion processing of bringing image quality close to image quality of an image of the first medical apparatus and an image of the second medical apparatus is read, and the parameter conversion processing of converting image quality on an image of the second medical apparatus that has been input is performed on the basis of the parameter conversion rule determined in advance.

### BRIEF DESCRIPTION OF DRAWINGS

FIG. 1 is a block diagram illustrating a configuration example of a first embodiment of a video processing system to which the present technology is applied.

FIG. 2 is a flowchart illustrating video processing for generating a conversion parameter.

FIG. 3 is a flowchart illustrating video processing for converting image quality of a medical video.

FIG. 4 is a block diagram illustrating a configuration example of a second embodiment of the video processing system to which the present technology is applied.

FIG. 5 is a block diagram illustrating a configuration example of an embodiment of a computer to which the present technology is applied.

FIG. 6 is a view illustrating an example of conversion processing of image quality of a medical video.

FIG. 7 is a view illustrating an example of parameter conversion processing of image quality of a medical video.

FIG. 8 is a diagram illustrating a configuration example of a surgical system to which the present technology is applied.

### MODE FOR CARRYING OUT THE INVENTION

Hereinafter, specific embodiments to which the present technology is applied will be described in detail with reference to the drawings.

### First Configuration Example of Video Processing System

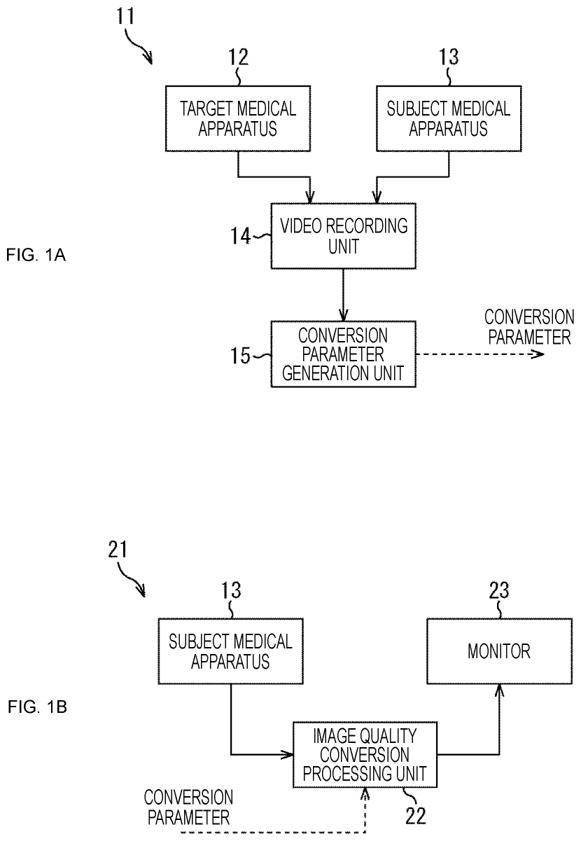

FIG. 1 is a diagram illustrating a configuration example of a first embodiment of a video processing system to which the present technology is applied.

A of FIG. 1 illustrates a first video processing system 11 being a side on which a conversion parameter to be used in image quality conversion processing is generated, and B of FIG. 1 illustrates a second video processing system 21 being a side on which image quality conversion processing is performed using the conversion parameter.

As illustrated in A of FIG. 1, the first video processing system 11 includes a target medical apparatus 12, a subject medical apparatus 13, a video recording unit 14, and a conversion parameter generation unit 15. The video recording unit 14 and the conversion parameter generation unit 15 are realized by a processor circuit included in one or more information processing devices. The information processing device is, for example, a computer.

The target medical apparatus 12 is a first medical apparatus, for example, a medical apparatus conventionally used in a medical institution, and captures a video with image quality which is target image quality of a video captured by the subject medical apparatus 13 close. Then, a video (hereinafter referred to as a medical video) captured and obtained by actually using the target medical apparatus 12 in surgery or the like in daily medical practice is supplied to the video recording unit 14 and recorded.

The subject medical apparatus 13 is a second medical apparatus, for example, a medical apparatus newly introduced into a medical institution, and captures a video to be subjected to image quality conversion. Then, similarly to the target medical apparatus 12, a medical video captured and obtained by using the subject medical apparatus 13 in surgery or the like in daily medical practice is supplied to the video recording unit 14 and recorded. The subject medical apparatus 13 is, for example, a medical apparatus of the same category as that of the target medical apparatus 12 and is a medical apparatus designed by a company different from the company that designed the target medical apparatus 12. Furthermore, the subject medical apparatus 13 is, for example, a successor model to the target medical apparatus 12 or a model in a different version of the target medical apparatus 12.

The video recording unit 14 records medical videos of the target medical apparatus 12 and the subject medical apparatus 13. For example, as the video recording unit 14, a video recording device of a video management system conventionally used in a medical institution can be used.

Using the medical videos of both the target medical apparatus 12 and the subject medical apparatus 13 recorded in the video recording unit 14, the conversion parameter generation unit 15 generates a conversion parameter for converting image quality so that the image quality of the medical video of the subject medical apparatus 13 approaches the image quality of the medical video of the target medical apparatus 12. For example, a computer connected to a video management system in a medical institution can be used as the conversion parameter generation unit 15. This computer is connected to the video recording device of the video management system, and can perform learning processing by accessing a video recorded in the video recording device.

Furthermore, the conversion parameter generation unit 15 learns a parameter of a generator model that converts the medical video of the subject medical apparatus 13 into a medical video similar to the characteristics of the target medical apparatus 12 by using a machine learning model, and uses the parameter acquired by the learning as the conversion parameter. The machine learning model is a model that learns a group of pairs of images including images of a first medical apparatus and images of a second medical apparatus and generates a parameter, and is, for example, a machine learning model that performs learning on the basis of a neural network having a plurality of layers and generates a parameter. An example thereof is CycleGAN. It is needless to say that the present invention is not limited to this technique, and the conversion parameter generation unit 15 may generate the conversion parameter by using another technique.

CycleGAN is a type of Generative Adversarial Network (GAN) algorithm, and is an algorithm that uses two types of image groups to perform image conversion parameter learning of converting an image of one image group into an image having characteristics similar to those of an image of the other image group. Note that details of the algorithm of CycleGAN are described in Non-Patent Document 1 below.
Non-Patent Document 1: J. Y. Zhu, T. Park, P. Isola, and A. A. Efros. Unpaired Image-to-Image Translation using Cycle-Consistent Adversarial Networks. In Proc. of IEEE International Conference on Computer Vision, 2017. https://arxiv.org/pdf/1703.10593.pdf

Then, the conversion parameter generated by the conversion parameter generation unit 15 is supplied to the image quality conversion processing unit 22 of the second video processing system 21. For example, as a method of supplying the conversion parameter from the conversion parameter generation unit 15 to the image quality conversion processing unit 22, a method of supplying the conversion parameter via an external memory, a method of connecting the conversion parameter generation unit 15 and the image quality conversion processing unit 22 via a network and performing transmission and reception by network communication, or the like can be adopted.

As illustrated in B of FIG. 1, the second video processing system 21 includes the subject medical apparatus 13, the image quality conversion processing unit 22, and a monitor 23. The image quality conversion processing unit 22 is realized by a circuit included in one or more information processing devices. The information processing device is, for example, a computer.

The subject medical apparatus 13 is a medical apparatus that is the same as or similar to that used in the first video processing system 11, and a medical video captured by the subject medical apparatus 13 is supplied to the image quality conversion processing unit 22. For example, as a method of supplying a medical video from the subject medical apparatus 13 to the image quality conversion processing unit 22, a method of directly connecting the subject medical apparatus 13 and the image quality conversion processing unit 22 with a video transmission cable, a method of connecting the subject medical apparatus 13 and the image quality conversion processing unit 22 to the video management system in the medical institution to virtually connect the subject medical apparatus 13 and the image quality conversion processing unit 22 by using a function of electrically freely changing connection between a large number of inputs and outputs, or the like can be adopted.

The image quality conversion processing unit 22 performs image quality conversion processing of bringing the image quality of the medical video of the subject medical apparatus 13 close to the image quality of the medical video of the target medical apparatus 12 by using the conversion parameter generated by the conversion parameter generation unit 15. For example, the image quality conversion processing unit 22 performs the image conversion processing by the generator model in which parameter setting based on the conversion parameter generated by the machine learning model used in the conversion parameter generation unit 15 is configured. The generator model performs conversion processing included in the machine learning model of the conversion parameter generation unit 15 in real time, and can execute the learned conversion processing by giving the learned parameter. As a result, the image quality of the image output from the second medical apparatus can be brought close to the image quality of the image output from the first medical apparatus.

For example, as the image quality conversion processing unit 22, a method of using a dedicated single device, a method of incorporating the function of image quality conversion processing in a receiver connected to a monitor for video display in the video management system in the medical institution, a method realized by software of a computer connected to the video management system, or the like can be adopted. In addition, the image quality conversion processing unit 22 may be realized by a circuit on an IP converter that is connected to a medical apparatus and converts a signal output from the medical apparatus into an Internet Protocol (IP) signal (for example, an Ethernet signal). Furthermore, for example, the image quality conversion processing unit 22 may be realized by a server connected to the IP converter on a network. Then, the image quality conversion processing unit 22 outputs, to the monitor 23, a medical video subjected to image quality conversion obtained as a result of performing the image quality conversion processing on the medical video supplied from the subject medical apparatus 13.

The monitor 23 displays the medical video subjected to the image quality conversion output from the image quality conversion processing unit 22. For example, as a method of connecting the image quality conversion processing unit 22 and the monitor 23, a method of directly connecting the image quality conversion processing unit 22 and the monitor 23 with a video transmission cable, a method of connecting the image quality conversion processing unit 22 and the monitor 23 to the video management system in the medical institution to virtually connect the image quality conversion processing unit 22 and the monitor 23 by using a function of electrically freely changing connection between a large number of inputs and outputs, or the like can be adopted.

The first video processing system 11 and the second video processing system 21 are configured as described above, and the medical video of the subject medical apparatus 13 whose image quality has been converted so as to be close to the image quality of the medical video of the target medical apparatus 12 can be displayed on the monitor 23. Here, the image quality converted by the image quality conversion processing unit 22 includes a color tone, a gradation, lightness, a contour enhancement degree, and the like of a video. The color tone is a parameter including at least one of hue, brightness, and saturation. Therefore, for example, even if a surgeon who has used the target medical apparatus 12 newly uses the subject medical apparatus 13, the surgeon can perform medical practice such as surgery in a manner similar to the conventional manner.

Furthermore, the first video processing system 11 can record medical videos of the target medical apparatus 12 and the subject medical apparatus 13 in the video recording unit 14 when the target medical apparatus 12 and the subject medical apparatus 13 are used in surgery or the like in daily medical practice, and generate the conversion parameter from the medical videos. Therefore, it is possible to greatly reduce labor, time, and the like required to generate the conversion parameter.

For example, in the technique disclosed in Patent Document 1 described above, while calculation of a conversion parameter for bringing the image quality close can be automatically performed, it takes manual labor to image a test object, and it is necessary to use both the medical apparatuses for a certain period of time for imaging the test object. Therefore, in a case where the medical apparatuses are always used for diagnosis, surgery, or the like, it is assumed that it is difficult to secure such labor and time.

In contrast, the first video processing system 11 can automatically calculate the conversion parameter for bringing the image quality of the subject medical apparatus 13 close to the image quality of the target medical apparatus 12 by using medical videos captured by the target medical apparatus 12 and the subject medical apparatus 13. Therefore, the first video processing system 11 does not need to perform imaging for calculating the conversion parameter with manual labor, and can use medical videos obtained when the target medical apparatus 12 and the subject medical apparatus 13 are usually used. Therefore, in the first video processing system 11, it is not necessary to stop use of the target medical apparatus 12 and the subject medical apparatus 13 for diagnosis, surgery, or the like in order to generate the conversion parameter. As a result, it is possible to bring the image quality of the subject medical apparatus 13 close to the image quality of the target medical apparatus 12 with as little manual labor as possible.

Even in a case where the same object is imaged under the conditions same as those for the first medical apparatus, the image quality of the second medical apparatus is often different from the image quality of the first medical apparatus. For example, even if the same object is imaged under the same conditions by using the endoscope designed by company A, the image quality such as color tone, gradation, and contour enhancement degree is different from those of the endoscope designed by company B. In particular, color tone design may vary greatly depending on the company that designed the medical apparatus. Due to this difference, when replacing a medical apparatus, a medical staff member needs to be concerned about the influence on surgery because the image quality is different from the conventional image quality.

In contrast, by the image quality conversion processing realized by the first video processing system 11 and the second video processing system 21, the image quality of the second medical apparatus approaches the image quality of the first medical apparatus, and the influence of the difference in image quality on surgery can be reduced. Moreover, in this image quality conversion processing, it is possible to generate a conversion parameter in an arbitrary combination of a plurality of medical apparatuses in a medical institution. That is, a conversion parameter can be generated by an arbitrary combination of the target medical apparatus 12 and the subject medical apparatus 13, and the image quality can be converted such that the image quality of the medical video of the desired subject medical apparatus 13 becomes close to the image quality of the desired target medical apparatus 12.

### Processing Example of Video Processing

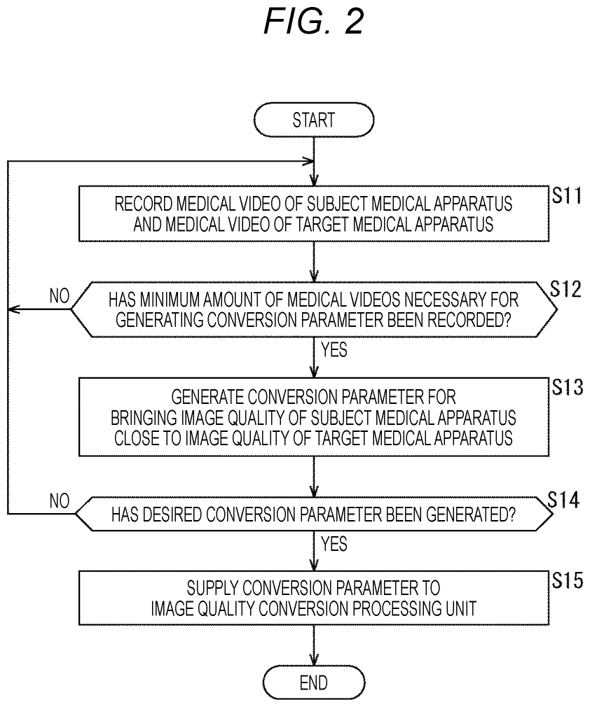

FIG. 2 is a flowchart illustrating video processing (processing of generating a conversion parameter) performed in the first video processing system 11.

In step S11, a medical video captured by the target medical apparatus 12 and a medical video captured by the subject medical apparatus 13 are recorded in the video recording unit 14. At this time, as described above, medical videos captured when the target medical apparatus 12 and the subject medical apparatus 13 are used in surgery or the like in daily medical practice are recorded.

In step S12, the conversion parameter generation unit 15 determines whether or not the minimum amount of medical videos necessary for generating a conversion parameter has been recorded in the video recording unit 14.

For example, the conversion parameter generation unit 15 determines whether or not a substantial recording time obtained by excluding a time required for preparation, a time in which a mainly intended object does not appear, and the like in the recording time of the medical video of the target medical apparatus 12 recorded in the video recording unit 14 reaches a prescribed time set in advance. Similarly, the conversion parameter generation unit 15 determines whether or not a substantial recording time of the recording time of the medical video of the subject medical apparatus 13 recorded in the video recording unit 14 reaches the prescribed time. Then, in a case where it is determined that the substantial recording time of both the medical videos of the target medical apparatus 12 and the subject medical apparatus 13 has reached the prescribed time, the conversion parameter generation unit 15 can determine that the minimum amount of medical videos necessary for generating the conversion parameter is recorded in the video recording unit 14.

In step S12, in a case where the conversion parameter generation unit 15 determines that the minimum amount of medical videos necessary for generating a conversion parameter is not recorded in the video recording unit 14, processing returns to step S11. That is, in this case, since the substantial recording time of the medical videos of both the target medical apparatus 12 and the subject medical apparatus 13 or the substantial recording time of one of the medical videos has not reached the prescribed time, recording of the medical videos in step S11 is continuously performed.

In contrast, in step S12, in a case where the conversion parameter generation unit 15 determines that the minimum amount of medical videos necessary for generating a conversion parameter is recorded in the video recording unit 14, processing proceeds to step S13.

In step S13, using the medical videos of both the target medical apparatus 12 and the subject medical apparatus 13 recorded in the video recording unit 14, the conversion parameter generation unit 15 generates a conversion parameter for converting the image quality so that the image quality of the medical video of the subject medical apparatus 13 approaches the image quality of the medical video of the target medical apparatus 12.

In step S14, it is determined whether or not a desired conversion parameter capable of obtaining a medical video with intended image quality has been generated.

For example, it is possible to use a technique of determining whether or not a desired conversion parameter is generated by a person visually recognizing whether or not the image quality of the video obtained by converting the video of the subject medical apparatus 13 for evaluation not used to generate the conversion parameter in step S13 by the generator model using the conversion parameter generated in step S13 is closer to the image quality of the video of the target medical apparatus 12. Note that, in addition to such a technique requiring manual labor, a technique using artificial intelligence for image quality evaluation or the like may be used, and determination as to whether or not a desired conversion parameter has been generated is not limited to a specific technique.

In a case where it is determined in step S14 that the desired conversion parameter has not been generated, the processing returns to step S11, and the similar processing is repeatedly performed and recording of the medical videos is continuously performed. In contrast, in a case where it is determined in step S14 that the desired conversion parameter has been generated, the processing proceeds to step S15.

In step S15, the conversion parameter generation unit 15 finally supplies the desired conversion parameter generated in step S13 to the image quality conversion processing unit 22, and thereafter, the processing is terminated.

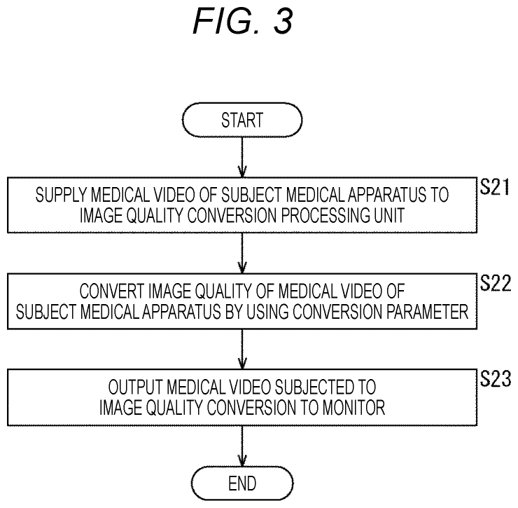

FIG. 3 is a flowchart illustrating video processing (processing of converting image quality of a medical video) performed in the second video processing system 21.

In step S21, the subject medical apparatus 13 supplies a medical video captured and obtained at the time of use in surgery or the like in medical practice to the image quality conversion processing unit 22.

In step S22, the image quality conversion processing unit 22 converts the image quality of the medical video supplied from the subject medical apparatus 13 in step S21 by using the conversion parameter supplied from the conversion parameter generation unit 15 in step S15 in FIG. 2. Therefore, image quality conversion processing is performed such that the image quality of the medical video captured by the subject medical apparatus 13 can be brought close to the image quality of the medical video captured by the target medical apparatus 12. Then, the image quality conversion processing unit 22 outputs the medical video subjected to image quality conversion to the monitor 23.

In step S23, the monitor 23 displays the medical video subjected to image quality conversion output from the image quality conversion processing unit 22 in step S22. Thereafter, the processing is terminated, and similar processing is performed every time a medical video is captured by the subject medical apparatus 13.

By the image processing as described above, a medical video of the subject medical apparatus 13 whose image quality has been converted so as to be close to the image quality of the medical video of the target medical apparatus 12 can be displayed on the monitor 23. As a result, even if the subject medical apparatus 13 is newly introduced into a medical institution, medical practice can be performed with the sense of use similarly to that of the conventional target medical apparatus 12.

### Second Configuration Example of Video Processing System

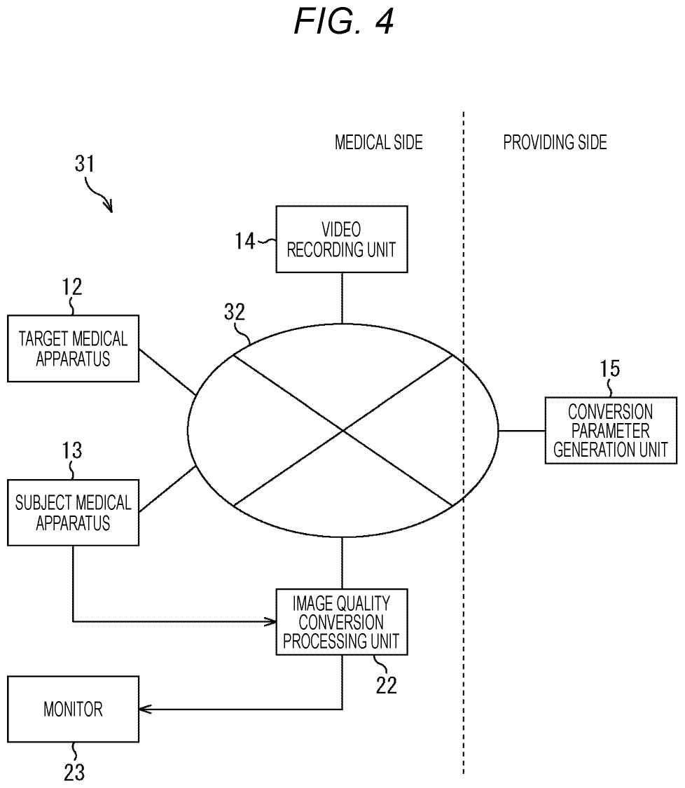

FIG. 4 is a block diagram illustrating a configuration example of a second embodiment of the video processing system to which the present technology is applied. Note that, in a video processing system 31 illustrated in FIG. 4, blocks common to the first video processing system 11 and the second video processing system 21 illustrated in FIG. 1 are denoted by the same reference numerals, and a detailed description thereof is omitted.

As illustrated in FIG. 4, the video processing system 31 is configured such that a target medical apparatus 12, a subject medical apparatus 13, a video recording unit 14, a conversion parameter generation unit 15, and an image quality conversion processing unit 22 are connected via a network 32. Furthermore, the video processing system 31 is configured such that a video is directly supplied from the subject medical apparatus 13 to the image quality conversion processing unit 22, and the video is directly output from the image quality conversion processing unit 22 to a monitor 23.

For example, the video processing system 31 can have a configuration in which the conversion parameter generation unit 15 is arranged in a place different from the place where the target medical apparatus 12, the subject medical apparatus 13, the video recording unit 14, the image quality conversion processing unit 22, and the monitor 23 are arranged. For example, in the video processing system 31, it is assumed that a business operator other than a medical institution provides a service for providing a conversion parameter, and the conversion parameter generation unit 15 is arranged on the service providing side. In contrast, the target medical apparatus 12, the subject medical apparatus 13, the video recording unit 14, the image quality conversion processing unit 22, and the monitor 23 are arranged on the medical institution side.

Therefore, the video processing system 31 can supply the medical video recorded in the video recording unit 14 to the conversion parameter generation unit 15 via the network 32, and supply the conversion parameter generated in the conversion parameter generation unit 15 to the image quality conversion processing unit 22. Therefore, also in the video processing system 31, similarly to the first video processing system 11 and the second video processing system 21 illustrated in FIG. 1, the medical video of the subject medical apparatus 13 whose image quality has been converted so as to be close to the image quality of the medical video of the target medical apparatus 12 can be displayed on the monitor 23.

Note that a medical video recorded in the video recording unit 14 may be copied to an external recording device such as a hard disk drive and moved to the service providing side without passing through the network 32, so that the medical video is supplied to the conversion parameter generation unit 15. Furthermore, similarly, the conversion parameter generated by the conversion parameter generation unit 15 may also be supplied to the image quality conversion processing unit 22 by using, for example, an external memory without passing through the network 32.

Furthermore, the video processing system 31 can be configured such that the target medical apparatus 12 and the subject medical apparatus 13 are arranged in different medical institutions. In this configuration, a video recording unit 14 for the target medical apparatus 12 and a video recording unit 14 for the subject medical apparatus 13 are prepared separately, and the medical videos are supplied from the respective video recording units 14 to the conversion parameter generation unit 15.

Note that, in the present embodiment described above, it has been described that the subject medical apparatus 13 newly introduced into the medical institution can capture a medical video and generate a conversion parameter when the subject medical apparatus 13 is used in actual medical practice. However, in a case where the subject medical apparatus 13 is a new product, it is assumed that there is a situation where the subject medical apparatus 13 cannot be used in actual medical practice and cannot capture a medical video. In this case, in lieu of the actual medical video of the subject medical apparatus 13, a video prepared for generating a conversion parameter by using the subject medical apparatus 13 is used. For example, it is assumed that a video obtained by performing imaging similar to actual use with the subject medical apparatus 13, a test video for evaluation at the time of product development of the subject medical apparatus 13, or the like is used.

Furthermore, a plurality of conversion parameter groups generated by using a plurality of groups of target medical apparatuses 12 may be generated in advance for a certain subject medical apparatus 13, and the image quality conversion processing unit 22 may switch between the plurality of conversion parameter groups and use one of the conversion parameter groups according to the surgeon who uses the subject medical apparatus 13, the clinical department, and the site to be observed. When the subject medical apparatus 13 is used in this manner, the image quality conversion processing unit 22 can hold the plurality of conversion parameter groups and select a desired conversion parameter when performing the image quality conversion processing. Alternatively, the plurality of conversion parameter groups may be stored in a different place from the image quality conversion processing unit 22, and a desired conversion parameter selected via the network 32 at the time of performing the image quality conversion processing can be transmitted to the image quality conversion processing unit 22.

Furthermore, the conversion parameter generated by using a combination of the target medical apparatus 12 which is specified and the subject medical apparatus 13 which is specified may be distributed or sold to be used for converting the image quality of a medical video captured by another solid medical apparatus (different from the subject medical apparatus 13 used for generating the conversion parameter) of the same model.

Furthermore, in the present embodiment described above, a mode has been described in which recording, learning, and conversion processing for bringing the image quality of the second medical apparatus (subject medical apparatus) close to the image quality of the first medical apparatus (target medical apparatus) are performed in a medical institution. However, recording of medical videos of the first medical apparatus and the second medical apparatus and learning using the medical videos require specialized knowledge and labor, and thus there is a high hurdle for a medical staff member to introduce the recording and the learning. Therefore, when the user selects the first medical apparatus and the second medical apparatus, a preset conversion parameter that is prepared in advance and brings the image quality of the second medical apparatus close to the image quality of the first medical apparatus may be set as the conversion parameter of the image quality conversion processing unit 22. As a result, a medical staff member can bring the image quality of the second medical apparatus close to the image quality of the first medical apparatus only by selecting the first medical apparatus and the second medical apparatus. Note that, in a case where the second medical apparatus and the image quality conversion processing unit cooperate with each other, since it is obvious that the second medical apparatus is selected, the user may select only the first medical apparatus. Furthermore, what the user selects may not be the model name of the first medical apparatus but may be the name or the number given for a parameter learned by using an image of the first medical apparatus and an image of the second medical apparatus. Furthermore, the preset conversion parameter may be stored in advance in the image quality conversion processing unit, or may be set by acquiring the preset conversion parameter downloaded from a server on the network.

Furthermore, the machine learning model may use only a specific area of an image as an area to be learned in the image of the first medical apparatus and the image of the second medical apparatus to be learned in machine learning. This is because, in an endoscopic image or the like, a black area derived from vignetting caused by an endoscope scope or an area on which a menu screen or the like generated by the endoscope system is superimposed is included in the image in some cases. In a case where machine learning is performed by using training data including these areas, it is conceivable that the learning is adversely affected. Therefore, the machine learning model may cut out an area of an image to be learned and use the cut out image as training data. For example, since the central area of a medical image is an area gazed by a medical staff member, the above-described black area or menu screen is rarely superimposed. Therefore, by setting only the central area of a medical image as an area to be learned, the image quality of the image of the second medical apparatus can be made close to the image quality of the image of the first medical apparatus. Note that the area to be learned is preferably automatically determined. For example, it is preferable that an area having a predetermined number of pixels from the center of an image is set as a learning area, or a rectangular area set in advance in a predetermined area of the image is set as a learning area. Furthermore, since a medical image has high resolution and a large data capacity, there is a case where machine learning is performed by dividing the medical image into a plurality of images when machine learning is performed. In such a machine learning model, processing of determining whether or not a divided image is appropriate as training data may be added. For example, in a case where a black area, an area in a color rarely seen in the body, or an area determined to be an overexposure area exists in a predetermined size or ratio or more, it may be determined that the area is inappropriate as an area to be learned, and processing of excluding the area from the training data may be added to the machine learning model.

Furthermore. The machine learning model may use, as an image to be learned, only an image captured in a specific surgical scene in the image of the first medical apparatus and the image of the second medical apparatus to be learned by the machine learning. For example, scene recognition processing based on metadata of a medical image or image recognition may be added to the machine learning model, and only a medical image of a predetermined scene may be set as the image to be learned. For example, since a scene in which it is determined that the scope of the endoscope is not inserted into the body may adversely affect machine learning, processing of determining that the scene is inappropriate as the scene to be learned and excluding the scene from the training data may be added to the machine learning model. Furthermore, in a case where metadata includes data indicating that imaging is different from normal imaging, such as zooming or superimposing a fluorescent image, the metadata may be excluded from the training data.

Furthermore, as the machine learning model, a machine learning model may be prepared for each image quality parameter. For example, learning may be performed by using separate learned models, that is, a first machine learning model that brings the color tone of the image of the second medical apparatus close to the color tone of the image of the first medical apparatus, a second machine learning model that brings the lightness of the image of the second medical apparatus close to the lightness of the image of the first medical apparatus, a third machine learning model that brings the contour enhancement of the image of the second medical apparatus close to the contour enhancement of the image of the first medical apparatus. At this time, the generator included in the image quality conversion processing unit is also set for each learned model. Therefore, the image quality of the image of the second medical apparatus can be brought closer to the image quality of the image of the first medical apparatus only with the image quality parameter selected by the user.

Furthermore, the machine learning model may include up-conversion or down-conversion processing of bringing the resolution of the image of the second medical apparatus close to the resolution of the image of the first medical apparatus in a case where the resolution of the image of the first medical apparatus is different from the resolution of the image of the second medical apparatus.

Furthermore, the machine learning model may perform machine learning for each observation mode by using training data for each observation mode, and a generator for each observation mode may be set in the image quality conversion processing unit. For example, a generator for each zoom magnification may be set in the image quality conversion processing unit. Furthermore, for example, in the image quality conversion processing unit, a generator may be set for each observation mode such as a normal light observation mode, a narrow band light observation mode, a near infrared light observation mode, and an ultraviolet light observation mode.

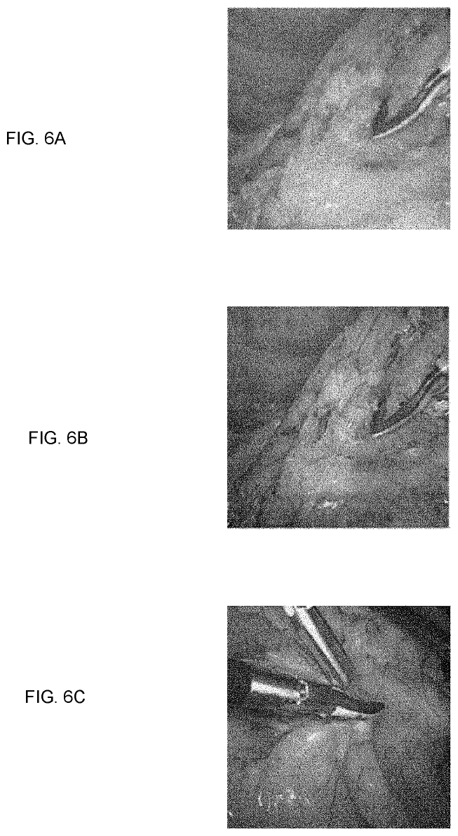

FIG. 6 is a view illustrating an example of conversion processing of image quality of a medical video. A of FIG. 6 is an image of the subject medical apparatus. B of FIG. 6 is a view illustrating an example in which the image quality conversion processing unit performs processing on the image of the subject medical apparatus. C of FIG. 6 is an image of the target medical apparatus. As illustrated in B of FIG. 6, the image quality of the image of the subject medical apparatus illustrated in A of FIG. 6 approaches the image quality of the target medical apparatus illustrated in C of FIG. 6. In particular, it can be seen that the color tone and contour enhancement of the image of the subject medical apparatus are changed, and the image quality of the image of the subject medical apparatus approaches the image quality of the image of the target medical apparatus. Therefore, with the video processing system to which the present technology is applied, when a medical staff member who prefers image quality of the first medical apparatus uses the second medical apparatus, the medical staff member can use the second medical apparatus with image quality close to image quality of the first medical apparatus.

### Third Configuration Example of Video Processing System

A third configuration example of the video processing system will be described. In the embodiment described above, the image quality conversion processing unit performs image quality conversion by using the generator in which a parameter based on the parameter generated by training the machine learning model is set. However, the image quality conversion by the generator of the machine learning model has a large calculation amount. Therefore, image quality conversion processing cannot be performed in real time, or a large amount of calculation resources is required. Furthermore, in a machine learning model such as Cycle-GAN, an object or partial deterioration that does not exist in the input image occurs in some cases, and it is difficult to guarantee the result of the image quality conversion processing. Therefore, in the third configuration example, in order to reduce the calculation amount of image quality conversion processing of the image conversion processing unit and easily guarantee the result of the image quality conversion processing, the image conversion processing unit performs parameter conversion processing based on a parameter conversion rule generated using a pair of images generated by the generator instead of the generator generated by the machine learning model.

The parameter conversion processing of the image conversion processing unit in the third configuration example will be described. The image conversion processing unit performs parameter conversion processing based on a parameter conversion rule generated by using a pair of images including an image (image close to the image quality of the image of the first medical apparatus) subjected to image quality conversion generated by the generator based on the parameter generated by training the machine learning model described above and an image (image of the second medical apparatus) before subjected to image quality conversion. The parameter conversion processing is processing of converting an image quality value (for example, RGB value, luminance, and emphasis level) of each pixel of the input image on the basis of a parameter conversion rule determined in advance. The parameter conversion rule determined in advance is, for example, a conversion rule using a lookup table that is generated by comparing a difference or the like between an image quality value of a pixel at a position determined in advance in an image before subjected to image quality conversion and an image quality value of a pixel at the same position in an image subjected to image quality conversion as a pair, and outputs a corresponding value when a value determined in advance is input. At this time, in order to cope with various situations, it is preferable to generate the parameter conversion rule by using an average value, a mode value, or the like of results of comparing values of each pixel in a plurality of pixel positions and a plurality of pairs of images. For example, the parameter conversion processing in the color tone is a 3D-LUT generated by using the average value of the differences between the RGB value of the image quality of the pixel at a position determined in advance in the image before subjected to the image quality conversion and the RGB value of the pixel at the same position in the image subjected to the image quality conversion as a pair.

At this time, in the 3D-LUT for converting the color tone, the number of pieces of data becomes very large when a corresponding color tone value for each color tone value is set. Therefore, it is preferable to reduce the table size by setting lattice points having a value range determined in advance. That is, it is preferable to generate the parameter conversion rule for each range of image quality values that is determined in advance on the basis of comparison between the pair of images described above. For example, it is preferable to generate a lookup table in which the R value is multiplied by 1.2 in a case where the R value falls within the range of 100 to 200 in the RGB values. Furthermore, it is preferable to set power of 2+1 lattice points. For example, by setting 17 points, the table can be equally divided into 16, and system processing such as memory processing becomes easy.

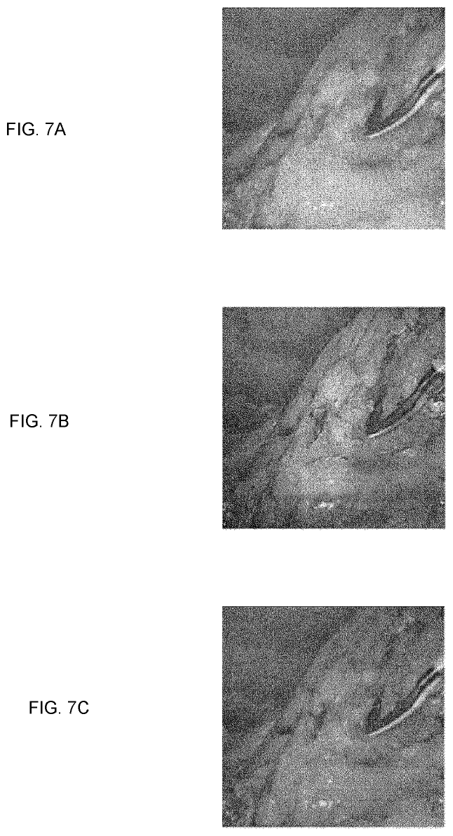

FIG. 7 is a view illustrating an example of parameter conversion processing of image quality of a medical video. A of FIG. 7 is an image of the subject medical apparatus. B of FIG. 7 is an image obtained by performing the image quality conversion processing on the image of the subject medical apparatus by using the machine learning model. C of FIG. 7 illustrates an image in which the image quality (color tone value) is converted by the parameter conversion rule generated on the basis of comparison between the image quality (color tone value) of each pixel of the image in A of FIG. 7 and the image quality (color tone value) of each pixel of the image in B of FIG. 7.

When the images illustrated in A of FIG. 7 and B of FIG. 7 are compared with each other, it can be seen that the color tone value and the contour value are converted as the image quality, and at the same time, noise is generated in the entire image. It is estimated that the noise is generated due to erroneous learning and low accuracy of the machine learning model (Cycle-GAN). In contrast, in the image illustrated in C of FIG. 7, since only the color tone value is converted according to the rule determined in advance, only the color tone is converted, and noise due to image quality conversion does not occur. Furthermore, since the rule for parameter conversion is clear, it is easy to guarantee the result. Furthermore, in the parameter conversion processing, since parameter conversion is performed on the input value by using the lookup table, the calculation amount is very small as compared with the case of using machine learning. Therefore, machine learning for generating a pair of images can be performed by a server having a large calculation resource, and parameter conversion processing can be performed in real time in an information processing device provided in an operating room.

Furthermore, the processing of bringing the image quality of the image of the second medical apparatus close to the image quality of the image of the first medical apparatus described in each of the above-described embodiments can also be applied to a surgical system.

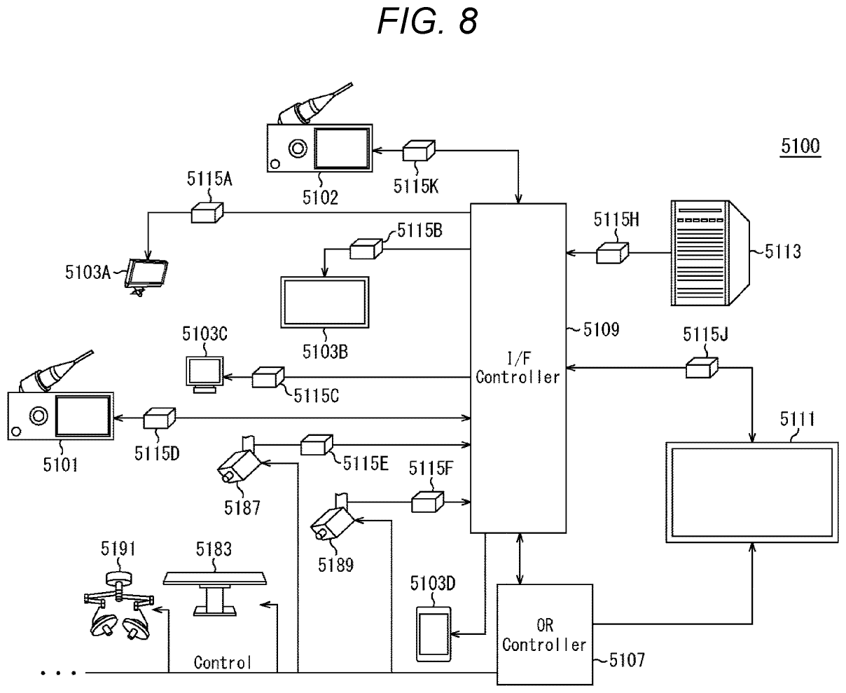

The surgical system will be described with reference to FIG. 8. A system 5100 is configured by connecting a group of devices installed in an operating room so as to be able to cooperate with each other via an operating room controller (OR Controller) 5107 and an input/output controller (I/F Controller) 5109. The operating room system 5100 is configured using an Internet Protocol (IP) network capable of transmitting and receiving 4K/8K videos, and transmits and receives input and output videos and control information for the devices via the IP network.

Various devices can be installed in the operating room. FIG. 8 illustrates, as an example, a first medical apparatus 5101 for endoscopic surgery, a second medical apparatus 5102 different from the first medical apparatus 5101, a ceiling camera 5187 provided on a ceiling of the operating room to image an operator's hand, an operating field camera 5189 provided on the ceiling of the operating room to image a state of the entire operating room, a plurality of display devices 5103A to 5103D, a patient bed 5183, and an illumination lamp 5191. Note that in addition to an endoscope illustrated in FIG. 8, various apparatuses for medical purposes that acquire images and videos, such as a master-slave endoscopic surgery robot and an X-ray imaging device, may be applied to the first medical apparatus 5101 and the second medical apparatus 5102.

The first medical apparatus 5101, the second medical apparatus 5102, the ceiling camera 5187, the operating field camera 5189, and the display devices 5103A to 5103C are connected to the I/F controller 5109 via IP converters 5115A to 5115F (hereinafter, denoted by reference numeral 5115 when not individually distinguished). The IP converters 5115D, 5115E, 5115F, and 5115K on video source sides (camera sides) perform IP conversion on videos from individual medical image capturing devices (such as an endoscope, an operation microscope, an X-ray imaging device, an operating field camera, and a pathological image capturing device), and transmit the results on the network. The IP converters 5115A to 5115D on video output sides (monitor sides) convert the videos transmitted through the network into monitor-unique formats, and output the results. Note that the IP converters on the video source sides function as encoders, and the IP converters on the video output sides function as decoders. The IP converters 5115 may have various image processing functions and functions as the image conversion processing unit described above. Furthermore, the IP converters 5115 may also have functions of, for example, resolution conversion processing corresponding to output destinations, rotation correction and image stabilization of an endoscopic video, and object recognition processing. Moreover, the IP converters 5115 may also include partial processing such as feature information extraction for analysis on a server described later. These image processing functions may be specific to the connected medical image devices, or may be upgradable from outside. The IP converters on the display sides can perform processing such as synthesis of a plurality of videos (for example, PinP processing) and superimposition of annotation information. Note that the protocol conversion function of each of the IP converters is a function to convert a received signal into a converted signal conforming to a communication protocol allowing the signal to be transmitted on the network (such as the Internet). Any communication protocol may be set as the communication protocol. Furthermore, the signal received by the IP converter and convertible in terms of protocol is a digital signal, and is, for example, a video signal or a pixel signal. Moreover, the IP converter may be incorporated in a video source side device or in a video output side device.

The first medical apparatus 5101 belongs to, for example, an endoscopic surgical system, and includes, for example, an endoscope and a display device that displays an image captured by the endoscope. Furthermore, the second medical apparatus 5102 is, for example, an endoscopic surgical system of a type different from that of the first medical apparatus. In contrast, the display devices 5103A to 5103D, the patient bed 5183, and the illumination lamp 5191 are, for example, devices equipped in the operating room separately from the endoscopic surgical system. Each of these apparatuses for surgery or diagnosis is also called an apparatus for medical purposes. The OR controller 5107 and/or the I/F controller 5109 controls operations of the apparatuses for medical purposes in cooperation. Similarly, in a case where a surgical robot (surgery master-slave) system and a medical image acquisition devices such as an X-ray imaging device are included in the operating room, those devices can also be connected as the first medical apparatuses 5101.

The OR controller 5107 controls processing related to image display in the apparatuses for medical purposes in an integrated manner. Specifically, the first medical apparatus 5101, the ceiling camera 5187, and the operating field camera 5189 among the devices included in the operating room system 5100 can each be a device (hereinafter, also referred to as a transmission source device) having a function to transmit information (hereinafter, also referred to as display information) to be displayed during surgery. Furthermore, the display devices 5103A to 5103D can each be a device (hereinafter, also called an output destination device) to output the display information. The OR controller 5107 has a function to control operations of the transmission source devices and the output destination devices so as to acquire the display information from the transmission source devices and transmit the display information to the output destination devices to cause the output destination devices to display or record the display information. Note that the display information includes various images captured during surgery, various information related to surgery (for example, physical information of a patient, information regarding past test results and surgical procedures, and the like), and the like.

Specifically, information regarding an image of a surgical site in a body cavity of the patient captured by the endoscope can be transmitted as the display information from the first medical apparatus 5101 to the OR controller 5107. Furthermore, information regarding an image of the area near the hands of the operator captured by the ceiling camera 5187 can be transmitted as the display information from the ceiling camera 5187. Moreover, information regarding an image representing the overall situation in the operating room captured by the operating field camera 5189 can be transmitted as the display information from the operating field camera 5189. Note that in a case where another device having an imaging function is present in the operating room system 5100, the OR controller 5107 may also acquire information regarding an image captured by the other device as the display information from the other device.

The OR controller 5107 displays the acquired display information (that is, an image captured during surgery and the various types of information regarding the surgery) on at least one of the display devices 5103A to 5103D serving as the output destination devices. In the illustrated example, the display device 5103A is a display device installed on the ceiling of the operating room, being hung therefrom; the display device 5103B is a display device installed on a wall surface of the operating room; the display device 5103C is a display device installed on a desk in the operating room; and the display device 5103D is a mobile apparatus (such as a tablet personal computer (PC)) having a display function.

The I/F controller 5109 controls input and output of the video signal from and to connected apparatuses. For example, the I/F controller 5109 controls input and output of the video signal on the basis of controlling of the OR controller 5107. The I/F controller 5109 includes, for example, an IP switcher, and controls high-speed transfer of the image (video) signal between apparatuses disposed on the IP network.

Furthermore, the operating room system 5100 may include a device outside the operating room. The device outside the operating room can be a server connected to a network built in and outside the hospital, a PC used by a medical staff member, or a projector installed in a meeting room of the hospital. In a case where such an external device is present outside the hospital, the OR controller 5107 can also display the display information on a display device of another hospital via, for example, a teleconference system for telemedicine.

Furthermore, an external server 5113 is, for example, an in-hospital server or a cloud server outside the operating room, and performs the above-described machine learning processing for converting the image quality of the image of the second medical apparatus to the image quality of the image of the first medical apparatus. Furthermore, the external server 5113 may be used for image analysis, data analysis, and the like. In this case, the video information in the operating room may be transmitted to the external server 5113, and the server may generate additional information through big data analysis or recognition/analysis processing using artificial intelligence (AI) (machine learning), and feed the additional information back to the display devices in the operating room. At this time, an IP converter 5115H connected to the video apparatuses in the operating room transmits data to the external server 5113, so that the video is analyzed. The transmitted data may be, for example, a surgical video itself captured by the endoscope or the like, metadata extracted from the video, data indicating an operating status of the connected apparatuses, or the like.

Moreover, the operating room system 5100 is further provided with a centralized operation panel 5111. Through the centralized operation panel 5111, a user can give the OR controller 5107 an instruction on input/output control of the I/F controller 5109 and an instruction on operation of the connected apparatuses. Furthermore, the user can switch image display via the centralized operation panel 5111. The centralized operation panel 5111 is configured by providing a touchscreen on a display surface of the display device. Note that the centralized operation panel 5111 may be connected to the I/F controller 5109 via an IP converter 5115J.

The IP network may be established using a wired network, or a part or the whole of the network may be established using a wireless network. For example, each of the IP converters on the video source sides may have a wireless communication function, and may transmit the received video to IP converters on the output side via a wireless communication network, such as the fifth-generation mobile communication system (5G) or the sixth-generation mobile communication system (6G).

### Configuration Example of Computer

Next, a series of processing (video processing method) described above can be performed by hardware or by software. In a case where the series of processing is performed by software, a program constituting the software is installed on a general-purpose computer, or the like.

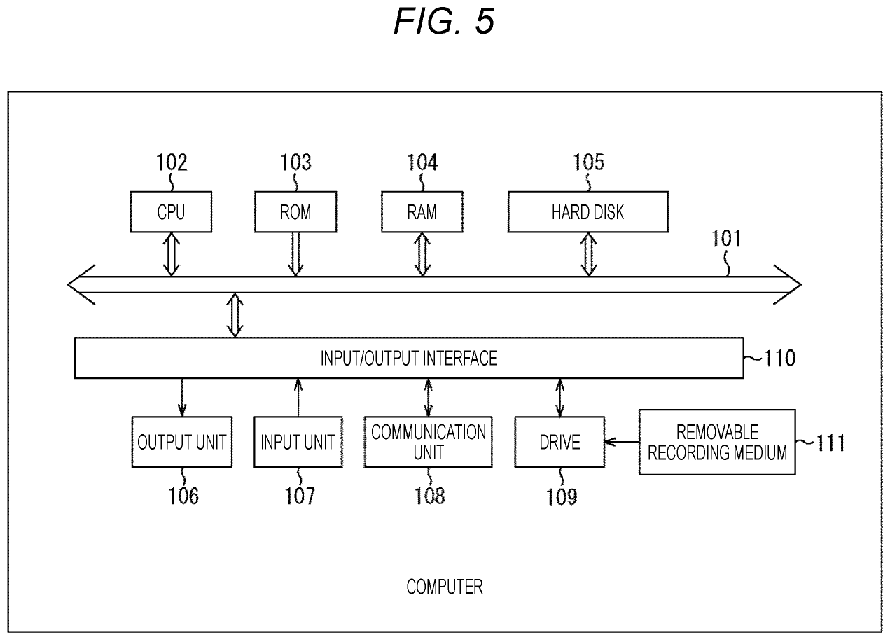

FIG. 5 is a block diagram illustrating a configuration example of an embodiment of a computer on which a program for executing the series of processing described above is installed.

The program can be recorded in advance on a hard disk 105 or a ROM 103 as a recording medium incorporated in the computer.

Alternatively, the program can also be stored (recorded) in a removable recording medium 111 driven by a drive 109. Such a removable recording medium 111 can be provided as so-called package software. Here, examples of the removable recording medium 111 include, for example, a flexible disk, a compact disc read only memory (CD-ROM), a magneto optical (MO) disk, a digital versatile disc (DVD), a magnetic disk, a semiconductor memory, and the like.

Note that, in addition to installing the program on the computer from the removable recording medium 111 as described above, the program can be downloaded to the computer via a communication network or a broadcasting network and installed on the incorporated hard disk 105. In other words, for example, the program can be wirelessly transferred from a download site to the computer via an artificial satellite for digital satellite broadcasting, or can be transferred by a wire to the computer via a network such as a local area network (LAN) or the Internet.

The computer has an incorporated central processing unit (CPU) 102, and an input/output interface 110 is connected to the CPU 102 via a bus 101.

When a command is input by the user, for example, operating an input unit 107 via the input/output interface 110, accordingly, the CPU 102 executes a program stored in the read only memory (ROM) 103. Alternatively, the CPU 102 loads a program stored in the hard disk 105 into a random access memory (RAM) 104 to execute the program.

Accordingly, the CPU 102 performs processing according to the flowchart described above or processing to be performed according to the configuration in the block diagram described above. Then, as necessary, the CPU 102 outputs a processing result from an output unit 106, or transmits the processing result from a communication unit 108, and further, causes the hard disk 105 to record the processing result, and the like, via the input/output interface 110, for example.

Note that, the input unit 107 includes a keyboard, a mouse, a microphone, and the like. Furthermore, the output unit 106 includes a liquid crystal display (LCD), a speaker, and the like.

Here, in the present description, the processing to be performed by the computer in accordance with the program is not necessarily performed in time series according to orders described in the flowcharts. In other words, the processing to be performed by the computer in accordance with the program includes processing to be executed in parallel or independently (for example, parallel processing or object-based processing). Furthermore, the program may be processed by one computer (one processor) or processed in a distributed manner by a plurality of computers. Moreover, the program may be transferred to a distant computer to be executed.

Moreover, in the present description, a system means a set of a plurality of components (devices, modules (parts), and the like), and it does not matter whether or not all the components are in the same housing. Consequently, a plurality of the devices housed in separate housings and connected via the network and one device in which a plurality of the modules is housed in one housing are both systems.

Furthermore, for example, a configuration described as one device (or one processing unit) may be divided and configured as a plurality of the devices (or processing units). Conversely, configurations described above as a plurality of devices (or processing units) may be collectively configured as one device (or processing unit). Furthermore, it goes without saying that a configuration other than the above-described configurations may be added to the configuration of each device (or each processing unit). Moreover, if the configuration and operation of the entire system are substantially the same, a part of the configuration of a certain device (or a certain processing unit) may be included in the configuration of another device (or another processing unit).

Furthermore, for example, the present technology can be configured as cloud computing in which one function is shared and jointly processed by a plurality of the devices via a network.

Furthermore, for example, the program described above can be executed by any device. In this case, the device is only required to have a necessary function (a functional block and the like) and obtain necessary information.

Furthermore, for example, each step described in the flowcharts described above can be executed by one device, or can be executed in a shared manner by a plurality of the devices. Moreover, in a case where a plurality of processes is included in one step, the plurality of the processes included in the one step can be executed by one device or shared and executed by a plurality of devices. In other words, the plurality of processes included in one step can also be executed as processes of a plurality of steps. On the contrary, the processes described as a plurality of steps can also be collectively executed as one step.

Note that, in the program to be executed by the computer, the processes in steps describing the program may be executed in time series in the order described in the present description, or may be executed in parallel, or independently at a necessary timing such as when a call is made. That is, as long as there is no contradiction, the processes of the respective steps may be executed in an order different from the above-described order. Moreover, the processes in the steps describing the program may be executed in parallel with processes of another program, or may be executed in combination with processes of the other program.

Note that, a plurality of the present technologies that has been described in the present description can each be implemented independently as a single unit unless there is a contradiction. Of course, a plurality of arbitrary present technologies can be implemented in combination. For example, a part or all of the present technologies described in any of the embodiments can be implemented in combination with a part or all of the present technologies described in other embodiments. Furthermore, a part or all of the present technologies described above may be implemented in combination with another technology not described above.

### Examples of Configuration Combinations

Note that the present technology may also have the following configurations.

(1)

A video processing system including:
a video recording unit that records a first medical video captured by a target medical apparatus, which is a first medical apparatus and a second medical video captured by a subject medical apparatus, which is a second medical apparatus;
a conversion parameter generation unit that generates a conversion parameter which brings image quality of the second medical video close to image quality of the first medical video by using the first medical video and the second medical video recorded in the video recording unit; and
an image quality conversion processing unit that converts image quality of a medical video captured by the subject medical apparatus by using the conversion parameter.
   (2)

The video processing system according to (1), in which
the first medical video is a video captured and obtained by actually using the target medical apparatus in daily medical practice, and
the second medical video is a video captured and obtained by actually using the subject medical apparatus in daily medical practice.
   (3)

The video processing system according to (1) or (2), in which
the target medical apparatus, the subject medical apparatus, the video recording unit, the conversion parameter generation unit, and the image quality conversion processing unit are connected via a network.
   (4)

The video processing system according to (3), in which
the conversion parameter generation unit is arranged on a providing side that performs a service for providing the conversion parameter separately from the target medical apparatus, the subject medical apparatus, the video recording unit, and the conversion parameter generation unit.
   (5)

The video processing system according to any one of (1) to (4), in which
a video prepared for generating the conversion parameter by using the subject medical apparatus is used as the second medical video.
   (6)

The video processing system according to any one of (1) to (5), in which
a plurality of the conversion parameters generated by using a plurality of the target medical apparatuses is generated in advance for the subject medical apparatus determined in advance; and
the image quality conversion processing unit switches between a plurality of the conversion parameters to convert image quality of a medical video captured by the subject medical apparatus determined in advance.
   (7)

The video processing system according to any one of (1) to (6), in which
the conversion parameter generated by using a combination of the target medical apparatus which is specified and the subject medical apparatus which is specified is used for conversion of image quality of a medical video captured by a medical apparatus that is another solid of a same model as the subject medical apparatus which is specified.
   (8)

A medical information processing system including:
one or more processors; and
a storage device that stores a program executed by the one or more processors, in which
the program is executed by the one or more processors
to read a parameter conversion rule determined in advance, the parameter conversion rule determined in advance being set on the basis of comparison between a pair of images including an image of a second medical apparatus subjected to image quality conversion processing of bringing image quality close to image quality of an image of a first medical apparatus and an image of the second medical apparatus, and
to perform parameter conversion processing of converting image quality on an image of the second medical apparatus that has been input on the basis of the parameter conversion rule determined in advance.
   (9)

The medical information processing system according to (8), in which
the parameter conversion rule determined in advance is a lookup table.
   (10)

The medical information processing system according to (8), in which
the parameter conversion rule determined in advance is a color tone 3D-LUT, and
the parameter conversion processing converts a color tone value on the basis of the 3D-LUT for each pixel of the image of the second medical apparatus that has been input.
   (11)

The medical information processing system according to any one of (8) to (10), in which
the pair of images is a pair of images including
an image generated by inputting an image of the second medical apparatus to a generator based on a parameter generated by a machine learning model that has learned an image of the first medical apparatus and an image of the second medical apparatus as training data, and
an image of the second medical apparatus that has been input.
   (12)

The medical information processing system according to (11), in which
the machine learning model is Cycle-GAN.
   (13)

The medical information processing system according to (8), in which
the one or more processors and the storage device are included in an Internet Protocol (IP) converter connected to the second medical apparatus.
   (14)

The medical information processing system according to (11), in which
the parameter conversion rule determined in advance is set on the basis of a result of comparing image quality of pixels at same positions in the pair of images.
   (15)

The medical information processing system according to (9), in which
the parameter conversion rule determined in advance is a lookup table that converts a RGB value determined in advance or an RGB value in a range determined in advance into a corresponding RGB value.
   (16)

An operation method of a medical information processing system including one or more processors, and a storage device that stores a program executed by the one or more processors, the operation method including:
by executing the program by the one or more processors,
reading a parameter conversion rule determined in advance, the parameter conversion rule determined in advance being set on the basis of comparison between a pair of images including an image of a second medical apparatus subjected to image quality conversion processing of bringing image quality close to image quality of an image of a first medical apparatus and an image of the second medical apparatus; and performing parameter conversion processing of converting image quality on an image of the second medical apparatus that has been input on the basis of the parameter conversion rule determined in advance.

Note that, the present embodiment is not limited to the embodiments described above, and various modifications can be made without departing from the gist of the present disclosure. Furthermore, the effects described herein are merely examples and are not limited, and other effects may be provided.

### REFERENCE SIGNS LIST

11 First video processing system
12 Target medical apparatus
13 Subject medical apparatus
14 Video recording unit
15 Conversion parameter generation unit
21 Second video processing system
22 Image quality conversion processing unit
23 Monitor
31 Video processing system
32 Network

1. A video processing system comprising:
a video recording unit that records a first medical video captured by a target medical apparatus, which is a first medical apparatus and a second medical video captured by a subject medical apparatus, which is a second medical apparatus;
a conversion parameter generation unit that generates a conversion parameter which brings image quality of the second medical video close to image quality of the first medical video by using the first medical video and the second medical video recorded in the video recording unit; and
an image quality conversion processing unit that converts image quality of a medical video captured by the subject medical apparatus by using the conversion parameter.

2. The video processing system according to claim 1, wherein
the first medical video is a video captured and obtained by actually using the target medical apparatus in daily medical practice, and
the second medical video is a video captured and obtained by actually using the subject medical apparatus in daily medical practice.

3. The video processing system according to claim 1, wherein
the target medical apparatus, the subject medical apparatus, the video recording unit, the conversion parameter generation unit, and the image quality conversion processing unit are connected via a network.

4. The video processing system according to claim 3, wherein
the conversion parameter generation unit is arranged on a providing side that performs a service for providing the conversion parameter separately from the target medical apparatus, the subject medical apparatus, the video recording unit, and the conversion parameter generation unit.

5. The video processing system according to claim 1, wherein
a video prepared for generating the conversion parameter by using the subject medical apparatus is used as the second medical video.

6. The video processing system according to claim 1, wherein
a plurality of the conversion parameters generated by using a plurality of the target medical apparatuses is generated in advance for the subject medical apparatus determined in advance, and
the image quality conversion processing unit switches between a plurality of the conversion parameters to convert image quality of a medical video captured by the subject medical apparatus determined in advance.

7. The video processing system according to claim 1, wherein
the conversion parameter generated by using a combination of the target medical apparatus which is specified and the subject medical apparatus which is specified is used for conversion of image quality of a medical video captured by a medical apparatus that is another solid of a same model as the subject medical apparatus which is specified.

8. A medical information processing system comprising:
one or more processors; and
a storage device that stores a program executed by the one or more processors, wherein
the program is executed by the one or more processors
to read a parameter conversion rule determined in advance, the parameter conversion rule determined in advance being set on a basis of comparison between a pair of images including an image of a second medical apparatus subjected to image quality conversion processing of bringing image quality close to image quality of an image of a first medical apparatus and an image of the second medical apparatus, and
to perform parameter conversion processing of converting image quality on an image of the second medical apparatus that has been input on a basis of the parameter conversion rule determined in advance.

9. The medical information processing system according to claim 8, wherein
the parameter conversion rule determined in advance is a lookup table.

10. The medical information processing system according to claim 8, wherein
the parameter conversion rule determined in advance is a color tone 3D-LUT, and
the parameter conversion processing converts a color tone value on a basis of the 3D-LUT for each pixel of the image of the second medical apparatus that has been input.

11. The medical information processing system according to claim 8, wherein
the pair of images is a pair of images including
an image generated by inputting an image of the second medical apparatus to a generator based on a parameter generated by a machine learning model that has learned an image of the first medical apparatus and an image of the second medical apparatus as training data, and
an image of the second medical apparatus that has been input.

12. The medical information processing system according to claim 11, wherein
the machine learning model is Cycle-GAN.

13. The medical information processing system according to claim 8, wherein
the one or more processors and the storage device are included in an Internet Protocol (IP) converter connected to the second medical apparatus.

14. The medical information processing system according to claim 11, wherein
the parameter conversion rule determined in advance is set on a basis of a result of comparing image quality of pixels at same positions in the pair of images.

15. The medical information processing system according to claim 9, wherein
the parameter conversion rule determined in advance is a lookup table that converts an RGB value determined in advance or an RGB value in a range determined in advance into a corresponding RGB value.

16. An operation method of a medical information processing system including one or more processors, and a storage device that stores a program executed by the one or more processors, the operation method comprising:
by executing the program by the one or more processors,
reading a parameter conversion rule determined in advance, the parameter conversion rule determined in advance being set on a basis of comparison between a pair of images including an image of a second medical apparatus subjected to image quality conversion processing of bringing image quality close to image quality of an image of a first medical apparatus and an image of the second medical apparatus; and
performing parameter conversion processing of converting image quality on an image of the second medical apparatus that has been input on a basis of the parameter conversion rule determined in advance.
